SEQUENCE LISTING (53 sequences)

SEQ ID NO: 1 - DNA, 341 bp, synthetic construct
  misc_feature 1..341: note = MonoS(H)SSS-HA
  source 1..341: mol_type = other DNA
agtagttcactagaagttgttgaagcgtcccccaccagcattcagatcagctgggatgctcctgctgtcacagtgcgttattaccgtatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgtacagaaattgacaaaccaagtcaaactagtaacggtcaaaactctggtactagttatccatatgatgttccaga

SEQ ID NO: 2 - DNA, 5131 bp, synthetic construct
  misc_feature 1..5131: note = pQ106-Ant-wt
  source 1..5131: mol_type = other DNA
ctcgagaaatcataaaaaatttatttgctttgtgagcggataacaattataatagattcaattgtgagcggataacaatttcacacagaattcattaaagaggagaaattacatatgcgtcatcaatctcatcaccatcaccatcacactagtggcactgagctcgagaacctgtactttcagggatcccaagatagtaccagcgatctgattccggcgccgccgctgagcaaggtgccgctgcagcaaaacttccaagacaaccagtttcagggtaaatggtacgttgttggtctggcgggcaacgcgatcctgcgtgaggacaaggacccgcagaaaatgtacgcgaccatttatgagctgaaggaagacaaaagctacaacgtgaccagcgttctgttccgtaagaagaaatgcgattattggattcgtaccttcgtgccgggtagccaaccgggcgaatttaccctgggtaacattaagagctacccgggcctgaccagctatctggttcgtgtggttagcaccaactataaccagcacgcgatggtgttctttaagaaagttagccaaaaccgtgagtacttcaagattaccctgtatggccgtaccaaggaactgaccagcgagctgaaagaaaacttcatccgttttagcaaaagcctgggtctgccggaaaaccacattgtgtttccggttccgatcgaccaatgcattgatggctaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 3 - DNA, 61 bp, synthetic construct
  misc_feature 1..61: note = SD8-MQANSGS-MonoS(H).F61
  source 1..61: mol_type = other DNA
ggattaaggaggtgatatttatgcaagccaattctggttctctagaagttgttgaagcgtc

SEQ ID NO: 4 - DNA, 62 bp, synthetic construct
  misc_feature 1..62: note = SD8-Lcn.F62
  source 1..62: mol_type = other DNA
ggattaaggaggtgatatttatgacaaatcgatctggttctcaagatagtaccagcgatctg

SEQ ID NO: 5 - DNA, 32 bp, synthetic construct
  misc_feature 1..32: note = MonoS(H)-GGG.R32
  source 1..32: mol_type = other DNA
tcctcctccaccttgacttggtttgtcaattt

SEQ ID NO: 6 - DNA, 33 bp, synthetic construct
  misc_feature 1..33: note = Lcn-GGG.R33
  source 1..33: mol_type = other DNA
tcctcctccaccgccatcaatgcattggtcgat

SEQ ID NO: 7 - DNA, 44 bp, synthetic construct
  misc_feature 1..44: note = T7SD8M2.F44
  source 1..44: mol_type = other DNA
atactaatacgactcactataggattaaggaggtgatatttatg

SEQ ID NO: 8 - DNA, 48 bp, synthetic construct
  misc_feature 1..48: note = MonoS(H)-VVN2.R48
  variation 25: note = n is a, c, g, or t
  variation 28: note = n is a, c, g, or t
  source 1..48: mol_type = other DNA
cccgcctcgcgcccgccgtccctanbbnbbtcctcctccaccttgact

SEQ ID NO: 9 - DNA, 49 bp, synthetic construct
  misc_feature 1..49: note = MonoS(H)-VVN2N1.R49
  variation 22: note = n is a, c, g, or t
  variation 26: note = n is a, c, g, or t
  variation 29: note = n is a, c, g, or t
  source 1..49: mol_type = other DNA
cccgcctcgcgcccgccgtccnctanbbnbbtcctcctccaccttgact

SEQ ID NO: 10 - DNA, 50 bp, synthetic construct
  misc_feature 1..50: note = Lcn-VVN2.R48
  variation 25: note = n is a, c, g, or t
  variation 28: note = n is a, c, g, or t
  source 1..50: mol_type = other DNA
cccgcctcgcgcccgccgtccctanbbnbbtcctcctccaccgccatcaa

SEQ ID NO: 11 - DNA, 51 bp, synthetic construct
  misc_feature 1..51: note = Lcn-VVN2N1.R49
  variation 22: note = n is a, c, g, or t
  variation 26: note = n is a, c, g, or t
  variation 29: note = n is a, c, g, or t
  source 1..51: mol_type = other DNA
cccgcctcgcgcccgccgtccnctanbbnbbtcctcctccaccgccatcaa

SEQ ID NO: 12 - DNA, 23 bp, synthetic construct
  misc_feature 1..23: note = Hex-Pu-an21-3
  source 1..23: mol_type = other DNA
cccgcctcgcgcccgccgtcccc

SEQ ID NO: 13 - DNA, 21 bp, synthetic construct
  misc_feature 1..21: note = an21-3.R21
  source 1..21: mol_type = other DNA
cccgcctcgcgcccgccgtcc

SEQ ID NO: 14 - DNA, 36 bp, synthetic construct
  misc_feature 1..36: note = SD8barcode1
  source 1..36: mol_type = other DNA
agtctaaggtaacggattaaggaggtgatatttatg

SEQ ID NO: 15 - DNA, 24 bp, synthetic construct
  misc_feature 1..24: note = MonoS(H)RealTimeR24
  source 1..24: mol_type = other DNA
actgtgacagcaggagcatcccag

SEQ ID NO: 16 - DNA, 20 bp, synthetic construct
  misc_feature 1..20: note = lcnrealtimer20
  source 1..20: mol_type = other DNA
cctgaaactggttgtcttgg

SEQ ID NO: 17 - DNA, 22 bp, synthetic construct
  misc_feature 1..22: note = MonoMidF22
  source 1..22: mol_type = other DNA
gcaagccaattctggttctcta

SEQ ID NO: 18 - DNA, 24 bp, synthetic construct
  misc_feature 1..24: note = MonoMidF22’
  source 1..24: mol_type = other DNA
cagcaagccaattctggttctcta

SEQ ID NO: 19 - DNA, 22 bp, synthetic construct
  misc_feature 1..22: note = LcnMidF22
  source 1..22: mol_type = other DNA
taccttcgtgccgggtagccaa

SEQ ID NO: 20 - DNA, 24 bp, synthetic construct
  misc_feature 1..24: note = LcnMidF22’
  source 1..24: mol_type = other DNA
agtaccttcgtgccgggtagccaa

SEQ ID NO: 21 - DNA, 30 bp, synthetic construct
  misc_feature 1..30: note = an21-3barcode11
  source 1..30: mol_type = other DNA
tcctcgaatccccgcctcgcgcccgccgtc

SEQ ID NO: 22 - DNA, 31 bp, synthetic construct
  misc_feature 1..31: note = an21-3barcode12
  source 1..31: mol_type = other DNA
ataggtggttccccgcctcgcgcccgccgtc

SEQ ID NO: 23 - DNA, 32 bp, synthetic construct
  misc_feature 1..32: note = an21-3barcode13
  source 1..32: mol_type = other DNA
gctctaacggaccccgcctcgcgcccgccgtc

SEQ ID NO: 24 - DNA, 30 bp, synthetic construct
  misc_feature 1..30: note = an21-3barcode14
  source 1..30: mol_type = other DNA
ttggagtgtccccgcctcgcgcccgccgtc

SEQ ID NO: 25 - DNA, 369 bp, synthetic construct
  misc_feature 1..369: note = MonoNNW1stProduct
  source 1..369: mol_type = other DNA
agtagttcactagaagttgttgaagcgtcccccaccagcattcagatcagctgggatgctcctgctgtcacagtgcgttattaccgtatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgtacagaaattgacaaaccaagtcaaactagtaacggtcaaaactctggtactagttatccatatgatgttccagattatgctggtggaggaggaggtagctag

SEQ ID NO: 26 - DNA, 62 bp, synthetic construct
  misc_feature 1..62: note = MoS-QANSGS.F62
  source 1..62: mol_type = other DNA
ggattaaggaggtgatatttatgcaagccaattctggttctctagaagttgttgaagcgtcc

SEQ ID NO: 27 - DNA, 49 bp, synthetic construct
  misc_feature 1..49: note = MoS-GR-T.R49
  source 1..49: mol_type = other DNA
cccgcctcgcgcccgccgtccactatcggcctcctcctccaccttgact

SEQ ID NO: 28 - DNA, 49 bp, synthetic construct
  misc_feature 1..49: note = MoS-GR-C.R49
  source 1..49: mol_type = other DNA
cccgcctcgcgcccgccgtccgctatcggcctcctcctccaccttgact

SEQ ID NO: 29 - DNA, 49 bp, synthetic construct
  misc_feature 1..49: note = MoS-RR-T.R49
  source 1..49: mol_type = other DNA
cccgcctcgcgcccgccgtccactatcgacgtcctcctccaccttgact

SEQ ID NO: 30 - DNA, 49 bp, synthetic construct
  misc_feature 1..49: note = MoS-GS-C.R49
  source 1..49: mol_type = other DNA
cccgcctcgcgcccgccgtccgctagctgcctcctcctccaccttgact

SEQ ID NO: 31 - DNA, 49 bp, synthetic construct
  misc_feature 1..49: note = MoS-GR2-C(C5).R49
  source 1..49: mol_type = other DNA
cccgcctcgcgcccgccgtccgctatctgcctcctcctccaccttgact

SEQ ID NO: 32 - DNA, 49 bp, synthetic construct
  misc_feature 1..49: note = MoS-GR2-T(C6).R49
  source 1..49: mol_type = other DNA
cccgcctcgcgcccgccgtccactatctgcctcctcctccaccttgact

SEQ ID NO: 33 - DNA, 49 bp, synthetic construct
  misc_feature 1..49: note = MoS-GT-C(C7).R49
  source 1..49: mol_type = other DNA
cccgcctcgcgcccgccgtccgctatgtgcctcctcctccaccttgact

SEQ ID NO: 34 - DNA, 49 bp, synthetic construct
  misc_feature 1..49: note = MoS-GS-C(C8).R49
  source 1..49: mol_type = other DNA
cccgcctcgcgcccgccgtccgctagctacctcctcctccaccttgact

SEQ ID NO: 35 - DNA, 49 bp, synthetic construct
  misc_feature 1..49: note = MoS-GA-C(C9).R49
  source 1..49: mol_type = other DNA
cccgcctcgcgcccgccgtccgctaagcacctcctcctccaccttgact

SEQ ID NO: 36 - DNA, 49 bp, synthetic construct
  misc_feature 1..49: note = MoS-AA-C(C10).R49
  source 1..49: mol_type = other DNA
cccgcctcgcgcccgccgtccgctatgccgctcctcctccaccttgact

SEQ ID NO: 37 - DNA, 49 bp, synthetic construct
  misc_feature 1..49: note = MoS-DS-C(C11).R49
  source 1..49: mol_type = other DNA
cccgcctcgcgcccgccgtccgctagctgtctcctcctccaccttgact

SEQ ID NO: 38 - DNA, 49 bp, synthetic construct
  misc_feature 1..49: note = MoS-RR-G(C12).R49
  source 1..49: mol_type = other DNA
cccgcctcgcgcccgccgtcccctagcgacgtcctcctccaccttgact

SEQ ID NO: 39 - DNA, 49 bp, synthetic construct
  misc_feature 1..49: note = MoS-PR-C(C13).R49
  source 1..49: mol_type = other DNA
cccgcctcgcgcccgccgtccgctaccgcggtcctcctccaccttgact

SEQ ID NO: 40 - DNA, 49 bp, synthetic construct
  misc_feature 1..49: note = MoS-SN-C(C14).R49
  source 1..49: mol_type = other DNA
cccgcctcgcgcccgccgtccgctaattgcttcctcctccaccttgact

SEQ ID NO: 41 - DNA, 49 bp, synthetic construct
  misc_feature 1..49: note = MoS-RE-G(C15).R49
  source 1..49: mol_type = other DNA
cccgcctcgcgcccgccgtcccctattcccgtcctcctccaccttgact

SEQ ID NO: 42 - DNA, 48 bp, synthetic construct
  misc_feature 1..48: note = MoS-G5S.R48
  source 1..48: mol_type = other DNA
cccgcctcgcgcccgccgtccctagctacctcctcctccaccttgact

SEQ ID NO: 43 - DNA, 48 bp, synthetic construct
  misc_feature 1..48: note = MoS-GR(C17).R48
  source 1..48: mol_type = other DNA
cccgcctcgcgcccgccgtccctatcggcctcctcctccaccttgact

SEQ ID NO: 44 - DNA, 48 bp, synthetic construct
  misc_feature 1..48: note = MoS-RR(C18).R48
  source 1..48: mol_type = other DNA
cccgcctcgcgcccgccgtccctatcgacgtcctcctccaccttgact

SEQ ID NO: 45 - DNA, 49 bp, synthetic construct
  misc_feature 1..49: note = MoS-GP-C(C19).R49
  source 1..49: mol_type = other DNA
cccgcctcgcgcccgccgtccgctacgggcctcctcctccaccttgact

SEQ ID NO: 46 - DNA, 300 bp, synthetic construct
  misc_feature 1..300: note = MoS-pool-1
  variation 52..69: note = n is a, c, g, or t
  variation 214..243: note = n is a, c, g, or t
  source 1..300: mol_type = other DNA
ctagaagttgttgaagcgtcccccaccagcattcagatcagctgggatgctnnnnnnnnnnnnnnnnnngtgcgttattaccgtatcacttacggagaaacaggaggaaatagccctgtccaggagttcactgtgcctgggagcaagtctacagctaccatcagcggccttaaacctggagttgattataccatcactgtgtatgctgtcactnnnnnnnnnnnnnnnnnnnnnnnnnnnnnnccaatttccattaattaccgtacagaaattgacaaaccaagtcaaggtggaggagga

SEQ ID NO: 47 - DNA, 107 bp, synthetic construct
  misc_feature 1..107: note = Peptide-pool(n=15)
  variation 45..89: note = n is a, c, g, or t
  source 1..107: mol_type = other DNA
atactaatacgactcactataggattaaggaggtgatatttatgnnnnnnnnnnnnnnnnnnnnnnnnnnnnnnnnnnnnnnnnnnnnntgtggaagttcaggtgga

SEQ ID NO: 48 - DNA, 49 bp, synthetic construct
  misc_feature 1..49: note = Lcn-GR-T.R49
  source 1..49: mol_type = other DNA
cccgcctcgcgcccgccgtccactatcggcctcctcctccaccgccatc

SEQ ID NO: 49 - DNA, 49 bp, synthetic construct
  misc_feature 1..49: note = Lcn-PR-C.R49
  source 1..49: mol_type = other DNA
cccgcctcgcgcccgccgtccgctaccgcggtcctcctccaccgccatc

SEQ ID NO: 50 - DNA, 48 bp, synthetic construct
  misc_feature 1..48: note = Lcn-G5S.R48
  source 1..48: mol_type = other DNA
cccgcctcgcgcccgccgtccctagctacctcctcctccaccgccatc

SEQ ID NO: 51 - DNA, 55 bp, synthetic construct
  misc_feature 1..55: note = Peptide-GR-T.R55
  source 1..55: mol_type = other DNA
cccgcctcgcgcccgccgtccactatcggcctcctcctccacctgaacttccaca

SEQ ID NO: 52 - DNA, 55 bp, synthetic construct
  misc_feature 1..55: note = Peptide-PR-C.R55
  source 1..55: mol_type = other DNA
cccgcctcgcgcccgccgtccgctaccgcggtcctcctccacctgaacttccaca

SEQ ID NO: 53 - DNA, 54 bp, synthetic construct
  misc_feature 1..54: note = Peptide-G5S.R54
  source 1..54: mol_type = other DNA
cccgcctcgcgcccgccgtccctagctacctcctcctccacctgaacttccaca

(19) United States
(12) Patent Application Publication
(10) Pub. No.: US 2025/0171770 A1
(43) Pub. Date: May 29, 2025

(54) METHOD FOR SCREENING FOR SEQUENCE THAT REGULATES DISPLAY EFFICIENCY IN POLYNUCLEOTIDE PRESENTATION METHOD

(71) Applicant: NATIONAL UNIVERSITY CORPORATION TOKAI NATIONAL HIGHER EDUCATION AND RESEARCH SYSTEM, Aichi (JP)

(72) Inventors: Hiroshi MURAKAMI, Aichi (JP); Shun UMEMOTO, Aichi (JP); Taishi KONDO, Aichi (JP); Gosuke HAYASHI, Aichi (JP); Tomoshige FUJINO, Aichi (JP)

(73) Assignee: NATIONAL UNIVERSITY CORPORATION TOKAI NATIONAL HIGHER EDUCATION AND RESEARCH SYSTEM, Aichi (JP)

(21) Appl. No.: 18/839,796

(22) PCT Filed: Feb. 21, 2023
(86) PCT No.: PCT/JP2023/006250

§ 371 Date: Aug. 20, 2024

(30) Foreign Application Priority Data
Feb. 22, 2022 (JP) 2022-025979

(51) Int. Cl. C12N 15/10

(52) U.S. Cl. CPC C12N 15/1072; C12N 15/1068

(57) ABSTRACT

An object of the present invention is to provide a method capable of more easily screening for, with higher accuracy, a sequence that regulates display efficiency in a polynucleotide display method. The problem is solved by a method for screening for a sequence that regulates display efficiency in a polynucleotide display method, the screening method including steps of: (a) binding a polynucleotide-polypeptide complex, which is obtained through translation by a cell-free translation system from a polynucleotide including a polypeptide encoding sequence and random sequences, to a solid phase via a modification substance on the polypeptide in the polynucleotide-polypeptide complex; and (b) selecting a random sequence by using, as an index, a ratio (enrichment factor) of appearance frequency of each random sequence in the polynucleotide-polypeptide complex bound to the solid phase with respect to appearance frequency of each random sequence in the polynucleotide.

### TECHNICAL FIELD

This invention relates to methods for screening sequences that regulate the display efficiency in polynucleotide display method and the like.

### BACKGROUND ART

Functional molecules with high affinity and specificity for certain targets are used as detection tools and candidates for therapeutic agents. As one of techniques for producing such new functional molecules (peptides and proteins), there is an evolutionary molecular engineering technique for acquiring functional molecules from a library having great diversity. In the evolutionary molecular engineering, diversity is an important factor in the evolutionary molecular engineering technique because the greater the diversity of a library to be initially used, the higher the probability of obtaining a functional molecule with high affinity.

The diversity of the library varies greatly depending on a method to be used. In a phage display method widely used as an evolutionary molecular engineering technique, since transformation is required, the diversity of the library is limited to about 10⁹ to 10¹¹. On the other hand, in a method using a cell-free translation system such as an mRNA display method (this method and a similar method may be collectively referred to as “polynucleotide display method” in the present specification), a library having a diversity of about 10¹³ can be constructed because E. coli transformation is not involved.

In the mRNA display method, proteins are displayed on the inherited mRNA via a puromycin linker (PuL), However, since it is necessary to covalently bind PuL to mRNA before the translation reaction, multi-step reactions such as transcription into mRNA, ligation with Pub, and translation reaction have to be separately performed. Therefore, the present inventors have improved the mRNA display method and developed a TRAP display method that enables simple and quick selection. In the TRAP display method, an mRNA-Protein complex can be automatically generated from a DNA template using a reconstructed cell-free translation system, which is a TRAP reaction system, excluding RF1. As a result, since the above-described multi-step reaction can be continuously performed, the time required for the selection has been successfully shortened.

The diversity in the TRAP display method is represented by the product of the mRNA concentration, the volume of a translation reaction solution, and display efficiency of the protein on mRNA. The upper limit of the mRNA concentration is the ribosome concentration (about 1 μM) in the translation reaction solution, and the translation reaction solution is expensive and the amount used is limited. Therefore, in order to improve diversity, improvement in display efficiency is required.

NPL 1 reports an attempt to obtain N-terminal and C-terminal sequences that improve display efficiency in an mRNA display method. In this prior study, a library was generated in which both the N-terminal and C-terminal sequences of VHH antibodies were randomized, followed by transcription into mRNA and display of proteins on mRNA in a cell-free translation system. Then, only the mRNA-Protein complex was recovered by gel purification to enrich the N-terminal and C-terminal sequences with high display efficiency. As a result of several cycles of this selection, a combination of N-terminal and C-terminal sequences that slightly increase (1.2 times) the display efficiency in the mRNA display method was obtained. In NPL 2, similarly to NPL 1, a library was generated in which the C-terminal sequence of scFy was randomized, followed by transcription into mRNA and display of proteins on mRNA in a cell-free translation system. From this library, only the mRNA-Protein complex was recovered using beads in which the target of the phenotypic scFv was immobilized to enrich the C-terminal sequence with high display efficiency. As a result, a C-terminal sequence that improves the display efficiency by about three times was obtained. However, these methods need to repeat selection before obtaining a sequence with high display efficiency, and versatility thereof is also unknown.

### CITATION LIST

### Non-Patent Literatures

NPL 1: K. Takahashi, M. Sunohara, T. Terai, S. Kumachi, N. Nemoto, Enhanced mRNA-protein fusion efficiency of a single-domain antibody by selection of mRNA display with additional random sequences in the terminal translated regions. Biophysics and Physicobiology 14, 23-28 (2017).

NPL 2: Y. Nagumo, K. Fujiwara, K. Horisawa, H. Yanagawa, N. Doi, PURE mRNA display for in vitro selection of single-chain antibodies. Journal of Biochemistry 159, 519-526 (2016).

### SUMMARY OF INVENTION

### Technical Problem

An object of the present invention is to provide a method capable of more easily screening for, with higher accuracy, a sequence that regulates display efficiency in a polynucleotide display method.

### Solution to Problem

As a result of intensive studies in view of the above problems, the present inventors have found that the above problems can be solved by a method for screening for a sequence that regulates display efficiency in a polynucleotide display method, the screening method including steps of: (a) binding a polynucleotide-polypeptide complex, which is obtained through translation by a cell-free translation system from a polynucleotide including a polypeptide encoding sequence and random sequences, to a solid phase via a modification substance on the polypeptide in the polynucleotide-polypeptide complex; and (b) selecting a random sequence by using, as an index, a ratio (enrichment factor) of appearance frequency of each random sequence in the polynucleotide-polypeptide complex bound to the solid phase with respect to appearance frequency of each random sequence in the polynucleotide. The present inventors have found a sequence that does not depend on the polypeptide encoding sequence and improves display efficiency in a polynucleotide display method by using the screening method. As a result of further studies based on these findings, the present inventors have completed the present invention, That is, the present invention includes the following embodiments.

Item 1. A method for screening for a sequence that regulates display efficiency in a polynucleotide display method, the screening method including steps of:
(a) binding a polynucleotide-polypeptide complex, which is obtained through translation by a cell-free translation system from a polynucleotide including a polypeptide encoding sequence and random sequences, to a solid phase via a modification substance on the polypeptide in the polynucleotide-polypeptide complex; and
(b) selecting a random sequence by using, as an index, a ratio (enrichment factor) of appearance frequency of each random sequence in the polynucleotide-polypeptide complex bound to the solid phase with respect to appearance frequency of each random sequence in the polynucleotide.

Item 2. The screening method according to item 1, wherein in step (b), a random sequence having a high enrichment factor is selected as a sequence that improves display efficiency.

Item 3, The screening method according to item 1 or 2, wherein in step (b), a random sequence having the top 10% of the enrichment factor is selected as a sequence that improves display efficiency.

Item 4. The screening method according to any one of items 1 to 3, wherein the modification substance is at least one selected from the group consisting of biotin, a peptide tag, and a substance containing a readily reactive group.

Item 5. The screening method according to any one of items 1 to 4, wherein the random sequence is arranged downstream side of the polypeptide encoding sequence.

Item 6. The screening method according to item 5, wherein a linker sequence is arranged between the random sequence and the polypeptide encoding sequence.

Item 7. The screening method according to item 5 or 6, wherein a stop codon is arranged adjacent to the random sequence or in the random sequence.

Item 8. A polynucleotide including a polypeptide encoding sequence or a polypeptide encoding sequence insertion site, a sequence 1 having 3 bases in length, a sequence 2 having 3 bases in length, and a stop codon, the polypeptide encoding sequence or polypeptide encoding sequence insertion site, the sequence 1, the sequence 2, and the stop codon being arranged in this order from upstream, the sequence 1 being adjacent to the sequence 2, and the sequence 1 being GGC, CGT, GGT, or AAA, and/or the sequence 2 being CGA, AGC, AGA, ACA, CGT, AGT, GCA, AGG, GGT, CGC, GCT, GGC, CGG, ACT, GAT, GAC, ATG, CCA, CCG, CCT, CTA, or CTG.

Item 9. The polynucleotide according to item 8, wherein the sequence 1 is GGC, CGT, GGT, or AAA, and the sequence 2 is CGA, AGC, AGA, ACA, CGT, AGT, GCA, AGG, GGT, CGC, GCT, GGC, CGG, ACT, GAT, GAC, ATG, CCA, CCG, CCT, CTA, or CTG.

Item 10. The polynucleotide according to item 8 or 9, wherein a contiguous sequence of the sequence 1 and the sequence 2 is GGCCGA, GGCAGA, GGCGCA, GGTAGA, GGCAGT, GGCAGC, GGCACA, GGCCGT, GGCGCT, GGCGAT, GGCACT, GGTCGA, GGCAGG, GGCACG, GGTGAC, GGTAGG, CGTCGA, GGTAGT, GGTCGT, GGCGGT, GGACGA, GGTGGT, GGTACA, GGTCGC, AATCGA, GGTAGC, or GCGCGA,

Item 11. The polynucleotide according to any one of items 8 to 10, wherein the sequence 2 and the stop codon are adjacent to each other.

Item 12. A reagent comprising the polynucleotide according to any one of items 8 to 11.

Item 13. The reagent according to item 12, which is a reagent for a polynucleotide display method.

### Advantageous Effects of Invention

According to the present invention, it is possible to provide a method capable of more easily screening for, with higher accuracy, a sequence that regulates display efficiency in a polynucleotide display method. According to the present invention, it is possible to provide a polynucleotide useful as a reagent for a polynucleotide display method, the polynucleotide comprising a sequence that does not depend on a polypeptide encoding sequence and improves display efficiency in a polynucleotide display method, Since an enrichment factor obtained from the N-terminal library reflects the efficiency of translation initiation, which is important in protein expression, it is considered that the protein expression level can be controlled by using this information. This is useful for production of useful substances using cells and high-efficiency production of proteins.

### BRIEF DESCRIPTION OF DRAWINGS

FIG. 1 shows a schematic view of a screening method for a C-terminal random sequence in Test Example 1. A: The design of the library used is shown. A random sequence was added to the C-terminus of DNA encoding Monobody or Anticalin. This random sequence was composed of Codon 1, Codon 2, and Base 1, and two types of mixed libraries of I and II were used. B: The procedure of the screening method used in this study is shown. A DNA library was prepared by PCR, and then was transcribed into mRNA, annealed with PuL, and subjected to a translation reaction in a TRAP reaction system. A portion of the library was recovered using a biotin-streptavidin interaction. The libraries before and after recovery were sequence-analyzed, and a ratio of appearance frequency in each library was calculated. This value was taken as an enrichment factor, and used as an index for predicting display efficiency.

FIG. 2A shows data analysis and evaluation of results of screening for the C-terminal random sequence of Test Example 1. A: A value in the Monobody library and a value in the Anticalin library were plotted for the enrichment factor of each C-terminal sequence. Each distribution chart is shown in the lower part. The positions of the sequences used in B were shown in the distribution chart.

FIG. 2B shows data analysis and evaluation of results of screening for the C-terminal random sequence of Test Example 1. B: The display efficiency of Monobody added with 19 types of C-terminal sequences listed in Table 1 was measured. The result of plotting a value of the display efficiency against the enrichment factor is the lower left figure. The error bar represents standard deviation (N=3). A method of calculating the display efficiency is described in the lower right.

FIG. 3 shows a display efficiency measurement result when three types of C-terminal sequences are added to an upstream sequence other than T Monobody. c1 is a C-terminal sequence located at the first enrichment factor, c16 is a C-terminal sequence located at the top 2.75%, and c13 is a C-terminal sequence located at 34.69%. The error bar represents standard deviation (N=3). A: Monobody library B: Anticalin C: Macrocyclic peptide library.

FIG. 4 shows a heat map (Test Example 1) in which the enrichment factor of each C-terminal sequence in the Monobody library is expressed by color.

FIG. 5 shows a schematic view of a screening method for an N-terminal random sequence in Test Example 3.

FIG. 6 shows data analysis of results of screening for the N-terminal random sequence of Test Example 3. A: To confirm the reliability, two independent experiments were repeated using the Monobody library, and the enrichment factor of each N-terminal sequence of the obtained data was plotted. B: A value of each N-terminal sequence of the data obtained for two types of sequences of Monobody and Anticalin was plotted for the enrichment factor of each N-terminal sequence, C: A value of each N-terminal sequence of the data obtained for two types of sequences including the SD sequence was plotted for the enrichment factor of each N-terminal sequence.

FIG. 7 shows a heat map (Test Example 4) in which the enrichment factor of each C-terminal sequence in the Monobody library is expressed by color.

### DESCRIPTION OF EMBODIMENTS

In the present specification, the expressions “comprise” and “contain” include the concepts of “comprise”, “contain”, “consist essentially of”, and “consist of”.

### 1. Screening Method

The present invention, in one embodiment thereof, relates to a method for screening for a sequence that regulates display efficiency in a polynucleotide display method (also sometimes referred to as the “screening method of the present invention” in the present specification) including steps (a) and (b). This will be described below.

Step (a) is a step of binding a polynucleotide-polypeptide complex, which is obtained through translation by a cell-free translation system from a polynucleotide (also sometimes referred to as the “polynucleotide for screening” in the present specification) including a polypeptide encoding sequence and random sequences, to a solid phase via a modification substance on the polypeptide in the polynucleotide-polypeptide complex.

The polypeptide encoding sequence included in the polynucleotide for screening is not particularly limited as long as it is a base sequence encoding a polypeptide. Examples of the polypeptide include antibodies, monobodies, enzymes, ligands, receptors, structural proteins, chain polypeptides, oligopeptides, and fragments thereof.

The random sequence included in the polynucleotide for screening is a sequence to be screened. The polynucleotide for screening is a mixture containing a plurality of polynucleotides each having a different random sequence. The number of types of the polynucleotide for screening (the number of types of random sequence) is, for example, 10¹ to 10¹¹. The lower limit of the number can be, for example, 10² or 10³. The upper limit of the number can be, for example, 10¹⁰, 10⁹, 10⁸, 10⁷, 10⁶, 10⁵, or 10⁴.

The position of the random sequence is not particularly limited. The random sequence can be arranged upstream of the polypeptide encoding sequence (that is, the N-terminal side when translated into a polypeptide) or can also be arranged downstream side of the polypeptide encoding sequence (that is, the C-terminal side when translated into a polypeptide). In a preferred embodiment of the present invention, the random sequence is arranged downstream side of the polypeptide encoding sequence. Thus, it is possible to screen for a sequence that can regulate display efficiency without depending on the type or sequence of the polypeptide encoded by the upstream polypeptide encoding sequence.

In the polynucleotide for screening, the random sequence may be present in only one site or in two or more sites that are not adjacent to each other. In one embodiment, a stop codon is included in the random sequence. In this case, a random sequence 1, a stop codon, and a random sequence 2 are arranged in this order.

The length of the random sequence (base length: the total length of all sites when the random sequence is present in two or more sites) is not particularly limited. The length is, for example, 3 to 200, preferably 3 to 100, more preferably 3 to 50, further preferably 4 to 30, still more preferably 5 to 20, and particularly preferably 5 to 10.

In the present specification, the “stop codon” is a codon that does not include a tRNA having a corresponding anticodon in the reaction system, and is not particularly limited to this. Specific examples of the stop codon include not only codons to which no amino acid is assigned on the codon table such as TAA, TAG, and TGA, but also codons corresponding to the anticodons of tRNA not added to the reaction system, and codons containing a modified base.

The configuration of the polynucleotide for screening is not particularly limited as long as the polypeptide encoded by the polypeptide encoding sequence can be synthesized by translation in a cell-free translation system. When the polypeptide encoding sequence does not have a start codon and/or a stop codon, the polynucleotide for screening has a start codon upstream of the polypeptide encoding sequence and/or a stop codon downstream of the polypeptide encoding sequence.

When the random sequence is arranged downstream side of the polypeptide encoding sequence in the polynucleotide for screening, another sequence is preferably arranged between the polypeptide encoding sequence and the random sequence. The other sequence can be preferably a linker sequence. The linker sequence is not particularly limited as long as it has a flexible chain structure, and examples thereof include a linker sequence containing glycine and/or serine. The length (base length) of the other sequence is, for example, 3 or more and preferably 4 or more. The upper limit of the length is not particularly limited, but can be, for example, 100, 50, 30, or 20.

As the cell-free translation system, both a prokaryotic cell translation system and a eukaryotic cell translation system can be used as the polynucleotide for screening. From the viewpoint of convenience, it is preferable to use a prokaryotic cell translation system as a cell-free translation system. When the polynucleotide for screening utilizes a prokaryotic cell translation system, it can preferably have a Shine-Dalgarno sequence (SD sequence) upstream of the start codon. When the polynucleotide for screening utilizes a eukaryotic cell translation system, it preferably has a Kozak sequence, a 5′ cap, and an internal ribosome entry site (IRES).

A preferred embodiment of the polynucleotide for screening includes a polynucleotide including a sequence in which a polypeptide encoding sequence, a random sequence, and a stop codon are arranged in this order from the upstream. In this embodiment, the random sequence and the stop codon are preferably adjacent to each other, and a linker sequence is preferably arranged between the polypeptide encoding sequence and the random sequence.

Another preferred embodiment of the polynucleotide for screening includes a polynucleotide including a sequence in which a start codon, a random sequence, a polypeptide encoding sequence, and a stop codon are arranged in this order from the upstream. In this embodiment, the start codon and the random sequence are preferably adjacent to each other.

The polynucleotide for screening can include a peptide tag encoding sequence adjacent to the polypeptide encoding sequence. In one embodiment, the peptide tag encoded by the peptide tag encoding sequence can be used as a modification substance on the polypeptide described later, and can be bound to a solid phase. Examples of the peptide tag include a His tag, a FLAG tag, a Halo tag, a GST tag, a MBP tag, a HA tag, a Myc tag, a V5 tag, and a PA tag.

The type of nucleotide constituting the polynucleotide for screening is not particularly limited as long as it can synthesize a polypeptide in a translation system. The polynucleotide for screening can be preferably RNA (mRNA).

The polynucleotide for screening can be obtained in accordance or compliance with a known method. For example, the polynucleotide for screening can be obtained by transcription in a cell-free transcription system from a DNA vector encoding a polynucleotide for screening.

The polynucleotide-polypeptide complex is obtained through translation by a cell-free translation system from the polynucleotide for screening in a cell-free translation system. The polynucleotide-polypeptide complex is a complex of a polynucleotide for screening and/or a complementary chain (for example, cDNA or the like) thereof and a polypeptide encoded by a polypeptide encoding sequence in the polynucleotide for screening. The complex can be obtained in accordance or compliance with a known method. For example, it is performed by binding puromycin downstream of the stop codon of the polynucleotide for screening. Puromycin may be bound to a polynucleotide for screening via a linker composed of a peptide or a nucleic acid. By binding puromycin to a downstream region of the stop codon of the polynucleotide for screening, the ribosome that has translated the polypeptide encoding sequence of the polynucleotide for screening takes up puromycin, and a complex of the polynucleotide for screening and the polypeptide is formed.

In the present specification, the “cell-free translation system” refers to a translation system that is free of cells, and as the cell-free translation system, an E. coli extract, a wheat germ extract, a rabbit red blood cell extract, an insect cell extract, and the like can be used. A reconstructed cell-free translation system constructed by reconstituting each of the purified ribosomal protein, aminoacyl-tRNA synthetase (aaRS), ribosomal RNA, amino acid, ERNA, GTP, ATP, translation initiation factor (IF) elongation factor (EF), termination factor (RF), ribosome regeneration factor (RRF), and other factors necessary for translation may be used. A system including RNA polymerase may also be used to perform transcription from DNA. As a commercially available cell-free translation system, RTS-100 (registered trademark) of Roche Diagnostics K.K. can be used as an E. coli-derived system, PURESYSTEM (registered trademark) of PGI and PURExpress® In Vitro Protein Synthesis Kit of New England BioLabs, or the like can be used as a reconstructed translation system, and those of ZOEGENE Corporation and CellFree Sciences can be used as a system using a wheat germ extract. As a system using ribosomes of E. coli, for example, a technique described in the following document is known: H. F. Kung et al., 1977. The Journal of Biological Chemistry Vol. 252, No. 19, 6889-6894; M. C. Gonza et al., 1985, Proceeding of National Academy of Sciences of the United States of America Vol. 82, 1648-1652; M. Y. Pavlov and M. Ehrenberg, 1996, Archives of Biochemistry and Biophysics Vol. 328, No. 1, 9-16; Y. Shimizu et al., 2001, Nature Biotechnology Vol. 19, No. 8, 751-755; H. Ohashi et al., 2007, Biochemical and Biophysical Research Communications Vol. 352, No. 1, 270-276. According to the cell-free translation system, an expression product can be obtained in a highly pure form without purification. Note that, the cell-free translation system of the present invention may be used not only for translation but also for transcription by adding a factor necessary for transcription.

The polynucleotide-polypeptide complex has, on the polypeptide, a modification substance for binding to a solid phase. The modification substance is not particularly limited as long as it can be used for binding to a solid phase, and examples thereof include biotin, a peptide tag, and a substance containing a readily reactive group, Biotin can bind to a solid phase modified with avidins (for example, streptavidin and the like). Depending on the type of the peptide tag, the peptide tag can bind to a solid phase modified with a substance having affinity with the peptide tag (for example, an anti-HA antibody in the case of an HA tag, a metal such as nickel in the case of a His tag, an anti-FLAG antibody in the case of a FLAG tag, and glutathione in the case of a GST tag). Examples of the readily reactive group include an ethynyl group, a vinyl group, an azide group, an epoxy group, an aldehyde group, and an oxylamino group. It is known that an ethynyl group forms a 1,2,3-triazole ring by a 1,3-dipolar cycloaddition reaction with an azide group. A vinyl group reacts with a thiol group to form a bond. An epoxy group reacts with an amino group or a thiol group to form a bond. An aldehyde group reacts with the amino group to form a Schiff base, when the Schiff base is reduced, a bond is formed. An oxylamino group reacts with a ketone group and an aldehyde group to form an oxime. It is known that an azide group forms a 1,2,3-triazole ring by a 1,3-dipolar cycloaddition reaction with an ethynyl group. These reactions can be used to bind a readily reactive group and a solid phase modified with a functional group corresponding thereto.

The modification substance can be bound to the polypeptide in accordance or compliance with a known method. For example, in the case of a peptide tag, as described above, the polypeptide can be modified with the peptide tag by arranging the peptide tag encoding sequence adjacent to the polypeptide encoding sequence in the polynucleotide for screening. In the case of a substance containing biotin or a readily reactive group, a tRNA obtained by acylating any tRNA with an amino acid containing these substances is introduced into a cell-free translation system, whereby the polypeptide can be modified with these substances.

Note that the tRNA can be prepared by using a flexizyme. The flexizyme is an artificial aminoacylated RNA catalyst capable of acylating any tRNA with any amino acid or hydroxy acid. When a flexizyme is used instead of an aminoacyl-tRNA synthesized by a natural aminoacyl-tRNA synthase, the genetic code table can be rewritten by matching a desired amino acid or hydroxy acid with an arbitrary codon. This is called codon reallocation. For codon reallocation, it is possible to use a translation system in which components of the translation system are freely removed according to a purpose and only necessary components are reconstructed. For example, when a translation system from which a specific amino acid has been removed is reconstructed, a codon corresponding to the amino acid becomes a free codon that does not encode any amino acid. Therefore, when an arbitrary amino acid is linked to a tRNA having an anticodon complementary to the free codon using a flexizyme or the like, and this is added to perform translation, the arbitrary amino acid is encoded by the codon, and a peptide into which the arbitrary amino acid has been introduced is translated instead of the removed amino acid.

The material for the solid phase is not particularly limited, and can be selected from, for example, an organic polymer compound, an inorganic compound, a biopolymer, and the like. Examples of the organic polymer compound include latex, polystyrene, and polypropylene. Examples of the inorganic compound include magnetic bodies (such as iron oxide, chromium oxide, and ferrite), silica, alumina, and glass. Examples of the biopolymer include insoluble agarose, insoluble dextran, gelatin, and cellulose. The material for the solid phase can be one kind alone, or may be a combination of two or more kinds thereof.

The shape of the solid phase is not particularly limited, and examples thereof include a particle, a microplate, a microtube, a test tube, and a membrane.

The binding of the polynucleotide-polypeptide complex to a solid phase can be performed in accordance or compliance with a known method depending on the type of the modification substance on the polypeptide, the type of the modification substance on the solid phase, and the like. Specifically, the polynucleotide-polypeptide complex and the solid phase are brought into contact with each other in a liquid having an appropriate composition, whereby the polynucleotide-polypeptide complex and the solid phase can be bound to each other. After the binding, it is preferable to perform washing with a solution or solvent in which the polynucleotide-polypeptide complex and the solid phase are not dissociated.

Step (b) is a step of selecting a random sequence by using, as an index, a ratio (enrichment factor) of appearance frequency of each random sequence in the polynucleotide-polypeptide complex bound to the solid phase with respect to appearance frequency of each random sequence in the polynucleotide for screening.

The appearance frequency (appearance frequency X) of each random sequence in the polynucleotide for screening can be, for example, a ratio of the number of molecules of the polynucleotide for screening having each random sequence with respect to:
(X1) the number of molecules of the polynucleotide for screening before being subjected to translation in a cell-free translation system,
(X2) the number of molecules of the polynucleotide for screening in a mixture (also including a polynucleotide for screening in which a polypeptide is not translated) containing a polynucleotide-polypeptide complex before being subjected to a binding operation to a solid phase, or
(X3) the number of molecules of the polynucleotide for screening in a mixture (also including a polynucleotide for screening in which a polypeptide is not translated) that has not been subjected to a solid-liquid separation operation after being subjected to a binding operation to a solid phase. For example, when the polynucleotide for screening is a mixture containing 10⁴ types of polynucleotides each having a different random sequence, and the number of molecules of a polynucleotide having a certain random sequence (random sequence A) in the mixture is 10², the appearance frequency X of the random sequence A is 1% (=(10²/10⁴)×100 (%)).

The appearance frequency (Appearance frequency Y) of each random sequence in the polynucleotide-polypeptide complex bound to the solid phase can be, for example, a ratio of the number of molecules of the polynucleotide for screening having each random sequence with respect to:
(Y1) the number of molecules of the polynucleotide for screening in the polynucleotide-polypeptide complex obtained through a solid-liquid separation operation after being subjected to a binding operation to a solid phase, or
(Y2) the number of molecules of the polynucleotide for screening in the polynucleotide-polypeptide complex obtained through a solid-liquid separation operation and a washing operation after being subjected to a binding operation to a solid phase.

The appearance frequency of each random sequence can be measured and calculated in accordance or compliance with a known method. The appearance frequency can be measured and calculated, for example, by sequence analysis using a next generation sequencer. In this case, the “number of molecules” described above can be replaced with the “number of reads” by a next generation sequencer.

In step (b), a random sequence is selected by using, as an index, a ratio (enrichment factor) of appearance frequency Y with respect to appearance frequency X. In the present invention, it has been found that the enrichment factor correlates with the display efficiency in a polynucleotide display method, and based on this, it has been found that a sequence that regulates display efficiency can be screened by step (b).

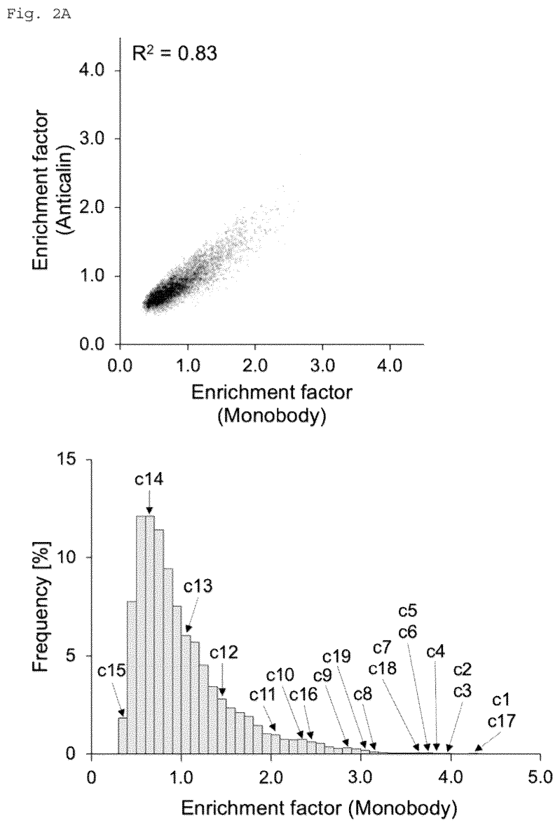

The polynucleotide display method is a method for artificially evolving a molecule by in vitro selection using a cell-free translation system. Examples of the polynucleotide display method include an mRNA display method, a cDNA display method, a ribosome display method, and a TRAP display method. The display efficiency can be measured in accordance or compliance with a known method, and for example, reaction product after translation from mRNA or the like encoding a protein to be presented in a cell-free translation system is electrophoresed, and the display efficiency can be measured from the band intensity, for example, according to the formula described in FIG. 2B described later.

In step (b), for example, by selecting a random sequence having a high enrichment factor, it is possible to screen for a sequence that improves display efficiency. Here, the “high enrichment factor” means a sequence having a higher enrichment factor when all random sequences are arranged in order of enrichment factor. In a preferred embodiment of the present invention, a random sequence having an enrichment factor of top 30% (preferably 20%, more preferably 10%, further preferably 5%, and still more preferably 3%) can be selected as a sequence that improves display efficiency, In another preferred embodiment of the present invention, a random sequence having an enrichment factor of 1.5 or more (preferably 2 or more, more preferably 2.5 or more, and further preferably 3 or more) can be selected as a sequence that improves display efficiency.

### 2. Polynucleotide and Reagent

The present invention, in one embodiment thereof, relates to a polynucleotide (also sometimes referred to as the “polynucleotide of the present invention” in the present specification) comprising a polypeptide encoding sequence or a polypeptide encoding sequence insertion site, a sequence 1 having 3 bases in length, a sequence 2 having 3 bases in length, and a stop codon, the polypeptide encoding sequence or polypeptide encoding sequence insertion site, the sequence 1, the sequence 2, and the stop codon being arranged in this order from upstream, the sequence 1 being adjacent to the sequence 2, and the sequence 1 being GGC, CGT, GGT, or AAA, and/or the sequence 2 being CGA, AGC, AGA, ACA, CGT, AGT, GCA, AGG, GGT, CGC, GCT, GGC, CGG, ACT, GAT, GAC, ATG, CCA, CCG, CCT, CTA, or CTG. The present invention, in one embodiment thereof, relates to a reagent (also sometimes referred to as the “reagent of the present invention” in the present specification) containing the polynucleotide of the present invention. These will be described below.

In a more preferred embodiment of the present invention, the sequence 1 is GGC, CGT, GGT, or AAA (or GGC, CGT, or GGT), and the sequence 2 is CGA, AGC, AGA, ACA, CGT, AGT, GCA, AGG, GGT, CGC, GCT, GGC, CGG, ACT, GAT, GAC, ATG, CCA, CCG, CCT, CTA, or CTG (or GA, AGC, AGA, ACA, CGT, AGT, GCA, AGG, GGT, CGC, GCT, GGC, CGG, ACT, GAT, or GAC).

In a more preferred embodiment of the present invention, a contiguous sequence of the sequence 1 and the sequence 2 is GGCCGA, GGCAGA, GGCGCA, GGTAGA, GGCAGT, GGCAGC, GGCACA, GGCCGT, GGCGCT, GGCGAT, GGCACT, GGTCGA, GGCAGG, GGCACG, GGTGAC, GGTAGG, CGTCGA, GGTAGT, GGTCGT, GGCGGT, GGACGA, GGTGGT, GGTACA, GGTCGC, AATCGA, GGTAGC, or GCGCGA.

In a still more preferred embodiment of the present invention, a contiguous sequence of the sequence 1 and the sequence 2 is GGCCGA, GGCAGA, GGTAGA, GGCACA, GGCCGT, GGCAGT, GGCAGC, GGCAGG, GGTCGA, GGCGCT, GGCACT, GGCGCA, or GGTAGG.

In a particularly preferred embodiment of the present invention, a contiguous sequence of the sequence 1 and the sequence 2 is GGCCGA, GGCAGA, GGTAGA, GGCCGT, GGCAGT, GGCAGC, GGTCGA, GGCGCT, or GGCACT.

In the polynucleotide of the present invention, it is desirable that the sequence 2 is arranged at a position relatively close to the stop codon, and the base length between the sequence 2 and the stop codon is preferably 0 to 6, more preferably 0 to 3, and further preferably 0 (that is, the sequence 2 and the stop codon are adjacent to each other). As a result, the display efficiency can be further improved.

In the polynucleotide of the present invention, one base adjacent to the downstream side of the stop codon is more preferably T or C.

The polypeptide encoding sequence insertion site can be, for example, a restriction enzyme site. Preferably, the polypeptide encoding sequence insertion site can be a multiple cloning site including a plurality of restriction enzyme sites.

The polynucleotide of the present invention can be, for example, mRNA, or can be a vector for expressing the mRNA.

As the configuration other than the above of the polynucleotide of the present invention, the configuration of the polynucleotide for screening described above can be adopted.

The reagent of the present invention is preferably a reagent for a polynucleotide display method. The polynucleotide display method is as described above.

In the reagent of the present invention, the polynucleotide of the present invention can be in the form of a composition containing the same. The composition may contain other components as necessary. Examples of the other components include a base, a carrier, a solvent, a dispersant, an emulsifier, a buffer, a stabilizer, an excipient, a binder, a disintegrant, a lubricant, a thickener, a moisturizing agent, a colorant, a fragrance, and a chelating agent.

The reagent of the present invention can be in the form of a kit. In this case, the reagent of the present invention (reagent kit) may contain an instrument, a reagent, or the like that can be used in a polynucleotide display method. Examples of the instrument include a test tube, a microplate, a particle, a latex particle, a column for purification, an epoxy coating slide glass, and a gold colloid coating slide glass. Examples of the reagent include a buffer, a cell-free transcription reagent, and a cell-free translation reagent.

### EXAMPLES

Hereinafter, the present invention will be described in detail based on Examples; however, the present invention is not limited by these Examples.

### Materials and Method

### (1) Sequence of Oligonucleotide and DNA Used

SEQ ID NO of sequences of oligonucleotide and DNA used in this study are shown in Table 1.

TABLE 1

| Name | SEQ ID NO |
|---|---|
| MonoS(H)SSS-HA | 1 |
| pQ106-Ant-wt | 2 |
| SD8-MQANSGS-MonoS(H).F61 | 3 |
| SD8-Len.F62 | 4 |
| MonoS(H)-GGG.R32 | 5 |
| Len-GGG.R33 | 6 |
| T7SD8M2.F44 | 7 |
| MonoS(H)-VVN2.R48 | 8 |
| MonoS(H)-VVN2N1.R49 | 9 |
| Len-VVN2.R48 | 10 |
| Len-VVN2N1.R49 | 11 |
| Hex-Pu-an21-3 | 12 |
| an21-3.R21 | 13 |
| SD8barcode1 | 14 |
| MonoS(H)RealTimeR24 | 15 |
| LenRealTimeR20 | 16 |
| MonoMidF22 | 17 |
| MonoMidF22 + 2 | 18 |
| LenMidF22 | 19 |
| LenMidF22 + 2 | 20 |
| an21-3barcode11 | 21 |
| an21-3barcode12 | 22 |
| an21-3barcode13 | 23 |
| an21-3barcode14 | 24 |
| MonoNNW1stProduct | 25 |
| MoS-QANSGS.F62 | 26 |
| MoS-GR-T.R49 | 27 |
| MoS-GR-C.R49 | 28 |
| MoS-RR-T.R49 | 29 |
| MoS-GS-C.R49 | 30 |
| MoS-GR2-C(C5).R49 | 31 |
| MoS-GR2-T(C6).R49 | 32 |
| MoS-GT-C(C7).R49 | 33 |
| MoS-GS-C(C8).R49 | 34 |
| MoS-GA-C(C9).R49 | 35 |
| MoS-AA-C(C10).R49 | 36 |
| MoS-DS-C(C11).R49 | 37 |
| MoS-RR-G(C12).R49 | 38 |
| MoS-PR-C(C13).R49 | 39 |
| MoS-SN-C(C14).R49 | 40 |
| MoS-RE-G(C15).R49 | 41 |
| MoS-G5S.R48 | 42 |
| MoS-GR(C17).R48 | 43 |
| MoS-RR(C18).R48 | 44 |
| MoS-GP-C(C19).R49 | 45 |
| MoS-pool-1 | 46 |
| Peptide-pool(n = 15) | 47 |
| Len-GR-T.R49 | 48 |
| Len-PR-C.R49 | 49 |
| Len-G5S.R48 | 50 |
| Peptide-GR-T.R55 | 51 |
| Peptide-PR-C.R55 | 52 |
| Peptide-G5S-R54 | 53 |

### (2) Preparation of DNA Library With C-Terminal Random Sequence

A PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM SD8-MQANSGS-MonoS (H).F61, 0.375 μM MonoS (H)-GGG.R32, 0.2 nM MonoS (H) SSS-HA, 2 nM Pfu-S DNA polymerase) was prepared, and a PCR reaction of 10 cycles was performed. This was designated as Template 1. Next, a PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM T7SD8M2.F44, 0.276 μM MonoS (H)-VVN2N1.R49 or 0.031 μM MonoS (H)-VVN1N2.R50, 0.375 nM Template 1, 2 nM Pfu-S DNA polymerase) was prepared, and a PCR reaction of 9 cycles was performed. The DNA after PCR was subjected to phenol chloroform treatment and isopropanol precipitation. This was designated as MonobodyVVNDNA. MonobodyNNNDNA was prepared in a similar manner as described above using MonoS (H)-NNN3.R53 instead of MonoS (H)-VVN2N1.R49.

A PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM SD8-Lcn.F62, 0.375 μM Lcn-GGG.R33, 0.375 nM pQ106-Ant-wt, 2 nM Pfu-S DNA polymerase) was prepared, and a PCR reaction of 18 cycles was performed. This was designated as Template 2. Next, a PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM T7SD8M2. F44, 0.276 μM Lcn-VVN2N1.R49 or 0.031 μM Lon-VVNIN2.R50, 0.375 nM Template 2, 2 nM Pfu-S DNA polymerase) was prepared, and a PCR reaction of 9 cycles was performed. The DNA after PCR was subjected to phenol chloroform treatment and isopropanol precipitation. This was designated as AnticalinVVNDNA.

### (3) Execution of Screening for C-Terminal Random Sequence

MonobodyVVNDNA, MonobodyNNNDNA, and AnticalinVVNDNA were transcribed into mRNA using T7 RNA polymerase, phenol chloroform treatment was performed, and isopropanol precipitation was preformed twice. These were designated as MonobodyVVNmRNA, MonobodyNNNmRNA, and AnticalinVVNmRNA, respectively. An annealing solution (6.67 μM MonobodyVVNmRNA or MonobodyNNNmRNA or AnticalinVVNmRNA, 4 μM Hex-PuL-an21-3, 25 mM HEPES-K pH7.8, 200 mM AcOK) was incubated at 95° C. for 3 minutes and at 25° C. for 5 minutes to prepare an mRNA-PuL complex. This was added to a reconstructed cell-free translation system (containing 16 μM Biotin-Phe-tRNA^{fMet}_{CAU} except for RF1 and Formyl donor) (mRNA-Pub complex: f.c. 1 μM) and incubated at 37° C. for 30 minutes. To this reaction solution (4 μL), 0.8 μL of 100 mM EDTA with pH 8.0 was added.

0.2 pmol of the mRNA-Protein complex prepared above was added to 20 μL of Dynabeads M-280 streptavidin (Thermo Fisher Scientific), and the mixture was rotationally mixed at 25° C. for 5 minutes. Then, after recovering the mRNA-Protein complex, a washing operation was performed with 50 μL of HBST (50 mM Hepes-KOH (pH 7.5), 300 mM NaCl, 0.05% (v/v) Tween 20) for 60 seconds, and a washing operation was performed with 50 μL of HBS (50 mM Hepes-KOH (pH 7.5), 300 mM NaCl) for 60 seconds.

0.02 pmol of a mRNA-Protein complex not subjected to a recovery operation (before recovery) and 10 μL of an annealing solution for RTprimer (5 μM an21-3.R21, 25 mM HEPES-K pH7.8, 200 mM AcOK) in total with respect to the total amount of a mRNA-Protein complex subjected to a recovery operation (after recovery) were added, and the mixture was incubated at 95° C. for 2 minutes and at 25° C. for 30 seconds. After recovery, only the supernatant after centrifugation was recovered. Thereafter, 10 μL of 2×RT mix (124 mM Tris-HCL (pH 8.4), 62.2 mM MgCl₂, 186 mM KCl, 13.7 mM dithiothreitol (DTT), 1.24 mM dNTPs, 0.8% (v/v) in-house moloney murine leukemia virus reverse transcriptase (HMLV)) was added, and a reverse transcription reaction was performed at 42° C. for 30 minutes.

To the reverse transcript, 180 μL of 1×PCR dNTPs (10 mM Tris-HCl pH 8.4, 100 mM KCl, 0.1% (v/v) Triton X-100, 2 mM MgSO₄, 0.22 mM dNTPs) was added. 85 μL of the resulting solution was added to an equal amount of a 2×PCR reaction solution (10 mM Tris-HCL pH 8.4, 100 mM KCl, 0.1% (v/v) Triton X-100, 4% (v/v) DMSO, 2 mM MgSO₄, 0.2 mM each dNTP, 4 nM Pfu-S DNA polymerase) and 1.28 μL of Primer mix [(25 μM MonoMidF22, 25 μM MonoMidF22+2, 50 μM an21-3barcode11 or an21-3barcode12) or (25 μM LcnMidF22, 25 μM LcnMidF22+2, 50 μM an21-3barcode13 or an21-3barcode14)], and a PCR reaction was performed for 12 cycles (13 cycles only after recovery of Anticalin). The DNA after PCR was subjected to phenol chloroform treatment and isopropanol precipitation. These DNAs were subjected to sequence analysis using a next generation sequencer (Macrogen Japan Corp.).

### (4) Calculation of Enrichment Factor

DNA read by sequence analysis was clustered for each C-terminal sequence, and the total number of reads was counted. The percentage of the number of reads of each C-terminal sequence with respect to the number of reads of the entire library was obtained as the appearance frequency, and the ratio of the appearance frequency after recovery to the appearance frequency before recovery was calculated as the enrichment factor. The distribution of the enrichment factor of the C-terminal sequence was prepared.

### (5) Preparation of mRNA Added With C-Terminal Sequence

Combinations of primers, template DNA, and cycle numbers used below are described in Table 2.

TABLE 2

| | 1st PCR | | | | 2nd PCR | | | |
|---|---|---|---|---|---|---|---|---|
| | Forward | Reverse | | No. of | Forward | Reverse | | No. of |
| | primer A | primer A | Template A | cycles (W) | primer B | primer B | Template B | cycles (2) |
| Monobody | MoS-QANSGS.F62 | MonoS(H)-GGG.R32 | [symbol] Product | 10 | [symbol] | MoS- [symbol] | 1st PCR product | 12 |
| WT | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | 12 |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| | | | | | | MoS- [symbol] | | |
| Monobody | MoS- [symbol] | MonoSMoS- [symbol] | MoS-MoS- [symbol] | 10 | [symbol] | MoS- [symbol] | 1st PCR product | 10 |
| library | | | | | | MoS- [symbol] | | |
| | | | | | | MoS- [symbol] | | |
| [symbol] | | | | | [symbol] | [symbol] | Template [symbol] | 8 |
| | | | | | | [symbol] | | |
| | | | | | | [symbol] | | |
| [symbol] | | | | | [symbol] | Peptide- [symbol] | Peptide- [symbol] | 10 |
| peptide | | | | | | Peptide- [symbol] | | |
| library | | | | | | Peptide- [symbol] | | |

[symbol]

indicates data missing or illegible when filed

A PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM Forward primer A, 0.375 μM Reverse primer A, 0.375 nM Template A, 2 nM Pfu-S DNA polymerase) was prepared, and a PCR reaction of W cycles was performed. Next, a PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM Forward primer B, 0.375 μM Reverse primer B, 0.375 nM Template B, 2 nM Pfu-S DNA polymerase) was prepared, and a PCR reaction of Z cycles was performed. The DNA after PCR was subjected to phenol chloroform treatment and isopropanol precipitation.

This was transcribed into mRNA using T7 RNA polymerase, phenol chloroform treatment was performed, and isopropanol precipitation was performed twice. Thereafter, gel purification was performed, and phenol chloroform treatment and isopropanol precipitation were performed again.

### (6) Display Efficiency Measurement

An annealing solution (4.8 μM mRNA, 4 82 M Hex-Pub-an21-3, 25 mM HEPES-K pH7.8, 200 mM AcOK) was incubated at 95° C. for 3 minutes and at 25° C. for 5 minutes to prepare an mRNA-PuL complex. This was added to a reconstructed cell-free translation system (except for RF1) (mRNA-PuL complex: f.c. 1 μM) and incubated at 37° C. for 30 minutes. However, for the macrocyclic peptide library, except for RF1 and Formyl donor, a reconstructed cell-free translation system newly added with 10 μM N-Chloroacetyl-L-Phe-tRNA Metu was used. The translation reaction with respect to Monobody library was performed at 37° C. for 10 minutes. In the case of measuring the display efficiency in DNA start, DNA (f.c. 5 nM) and Hex-PuL-an21-3 (f.c. 1 μM) were added to a reconstructed cell-free translation system (containing T7 RNA polymerase except for RF1) and incubated at 37° C. for 30 minutes.

To 1 μL of samples before and after the translation reaction, 11 μL of a gel loading buffer (62.5 mM Tris-HCl pH 6.8, 5 mM DIT, 0.05% (w/v) SDS, 10 mM MgCl₂, 20% (v/v) Glycerol) was added, and 2 μL thereof was electrophoresed by 8% PAGE (0.375 M Tris-HCl pH 8.8, 6 M Urea, 0.05% SDS). However, 8% PAGE (0.45 M Tris-HCl pH 8.8, 6 M Urea, 0.05% SDS) was used for the macrocyclic peptide library. The band was confirmed by fluorescence observation of HEX using ChemiDoc MP Imaging System (Bio-rad). From the band intensity present in the lane after the translation reaction, the display efficiency was calculated as shown in FIG. 2B.

### (7) Preparation of DNA Library With N-Terminal Random Sequence

A PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM MonoS (H) SSS. F26, 0.375 μM HATag-G4S.R48, 0.2 nM MonoS (H) SSS-HA, 2 nM Pfu-S DNA polymerase) was prepared, and a PCR reaction of 10 cycles was performed. This was designated as Template 3. Next, a PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 M MonoS (H) NNW3SSS. F58, 0.375 μM G5S-4Gan21-3. R42, 0.375 nM Template 3, 2 nM Pfu-S DNA polymerase) was prepared, and a PCR reaction of 9 cycles was performed. This was designated as Template 4. Next, a PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM T7SD8M2.F44, 0.375 μM G5S-4Gan21-3.R42, 0.375 nM Template 4, 2 nM Pfu-S DNA polymerase) was prepared, and a PCR reaction of 10 cycles was performed. The DNA after PCR was subjected to phenol chloroform treatment and isopropanol precipitation. This was designated as MonobodyNNWDNA. AnticalinNNWDNA was also prepared in a similar manner.

A PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM catMonoS (H) NNW3SSS. F60, 0.375 μM G5S-4Gan21-3.R42, 0.375 nM Template 3, 2 nM Pfu-S DNA polymerase) was prepared, and a PCR reaction of 9 cycles was performed. This was designated as Template 5. Next, a PCR reaction solution (10 mM Tris-HCl pH 8.4, 100 mM KCl, 0.1% (v/v) Triton X-100, 2% (v/v) DMSO, 2 mM MgSO₄, 0.2 mM each dNTP, 0.375 μM T7SDCATM. F46, 0.375 μM G5S-4Gan21-3.R42, 0.375 nM Template 4, 2 nM Pfu-S DNA polymerase) was prepared, and a PCR reaction of 10 cycles was performed. The DNA after PCR was subjected to phenol chloroform treatment and isopropanol precipitation. This was designated as catMonobodyNNWDNA.

### (8) Execution of Screening for N-Terminal Random Sequence

MonobodyNNWDNA, catMonobodyNNWDNA, and AnticalinNNWDNA were transcribed into mRNA using T7 RNA polymerase, phenol chloroform treatment was performed, and isopropanol precipitation was preformed twice. These were designated as MonobodyNNWmRNA, catMonobodyNNWmRNA, and AnticalinNNWDNA, respectively. An annealing solution (6.67 μM MonobodyNNWmRNA or catMonobodyNNWmRNA or AnticalinNNWDNA, 4 μM Hex-PuL-an21-3, 25 mM HEPES-K pH7. 8, 200 mM AcOK) was incubated at 95° C. for 3 minutes and at 25° C. for 5 minutes to prepare an mRNA-PuL complex. This was added to a reconstructed cell-free translation system (except for RFI) (mRNA-PuL complex: f.c. 1 μM) and incubated at 37° C. for 30 minutes. To this reaction solution (4 μL), 0.8 μL of 100 mM EDTA with pH 8.0 was added. 2.4 μL of 3×RT mix (150 mM Tris-HCl (pH 8.4), 75 mM MgCl₂, 225 mM KCl, 16.5 mM dithiothreitol (DTT), 1.5 mM dNTPs, 0.12% (v/v) in-house moloney murine leukemia virus reverse transcriptase (HMLV), 7.5 μM G4S.R19) was added thereto, and a reverse transcription reaction was performed at 42° C. for 15 minutes. Thereafter, filtration was performed using Zeba (trademark) Spin Desalting Columns 7K MWCO (Thermo Scientific) equilibrated with HBST (50 mM Hepes-KOH (pH 7.5), 300 mM NaCl, 0.05% (v/v) Tween 20), and buffer exchange was performed.

3.2 pmol of the mRNA-Protein complex after reverse transcription prepared above and 1 μL of Anti-HA tag mAb (TANA2) (Medical & Biological Laboratories Co., Ltd) were mixed and incubated at 25° C. for 30 minutes. 20 μL of Dynabeads (trademark) Protein G for immunoprecipitation (Thermo Fisher Scientific) was added thereto, and the mixture was rotationally mixed at 25° C. for 10 minutes. Then, after recovering the mRNA-Protein complex, a washing operation was performed with 10 μL of HBST (50 mM Hepes-KOH (pH 7.5), 300 mM NaCl, 0.05% (v/v) Tween 20) for 10 seconds.

0.4 pmol of a mRNA-Protein complex not subjected to a recovery operation (before recovery) and 1000 μL of 1×PCR dNTPS (10 mM Tris-HCl pH 8.4, 100 mM KCl, 0.1% (v/v) Triton X-100, 2 mM MgSO₄, 0.22 mM dNTPs) in total with respect to the total amount of a mRNA-Protein complex subjected to a recovery operation (after recovery) were added. 85 μL of the resulting solution was added to an equal amount of a 2×PCR reaction solution (10 mM Tris-HCl pH 8.4, 100 mM KCL, 0.1% (v/v) Triton X-100, 4% (v/v) DMSO, 2 mM MgSO₄, 0.2 mM each dNTP, 4 nM Pfu-S DNA polymerase) and 1.275 μL of Primer mix [(50 μM SD8barcode1 or SD8barcode2, 12.5 μM HATagR23+3, 12.5 μM HATagR23+2, 12.5 μM HATagR23+1, 12.5 μM HATagR23) or (50 μM SDcatbarcode9 or SDcatbarcode10, 12.5 μM HATagR23+3, 12.5 μM HATagR23+2, 12.5 μM HATagR23+1, 12.5 μM HATagR23)], and a PCR reaction was performed for 8 cycles. The DNA after PCR was subjected to phenol chloroform treatment and isopropanol precipitation. These DNAs were subjected to sequence analysis using a next generation sequencer (Macrogen Japan Corp.).

### Test Example 1. Data Analysis and Evaluation 1 of Screening Results for C-terminal Random Sequence

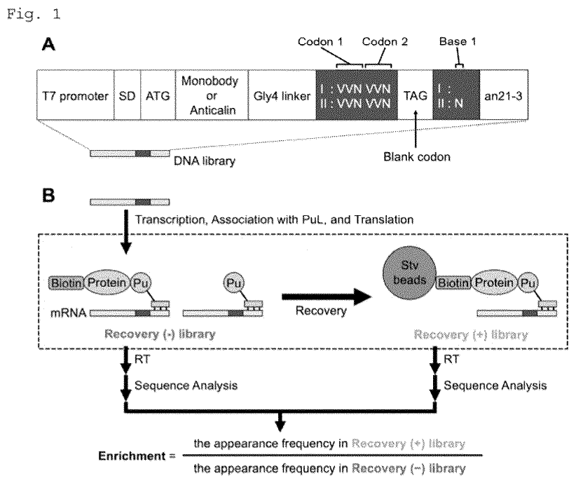

As shown in FIG. 1A, a mixed DNA library was prepared in which C-terminal random sequences of type I (VVNVVNTAG) and type II (VVNVVNTAGN) was added to an upstream sequence (Monobody or Anticalin). This DNA library was used as a template to be transcribed into mRNA, and an mRNA-Protein complex was prepared in a TRAP reaction system. This complex can be recovered with streptavidin beads using biotin modified at the N-terminus of the protein. The reverse transcription reaction to cDNA was performed on both the libraries before and after recovery, and sequence analysis was performed with a next generation sequencer. As a result of sequence analysis, 20 million or more reads could be acquired for each library. There are theoretically 7056 types of sequences in the mixed library, but at least one or more reads could be acquired for all sequences. Among them, sequences 100 or more reads both before and after recovery were made effective sequences for future analysis (99.9% or more of the whole excluding four kinds of Monobody, and 99.8% or more of the whole excluding nine kinds of Anticalin).

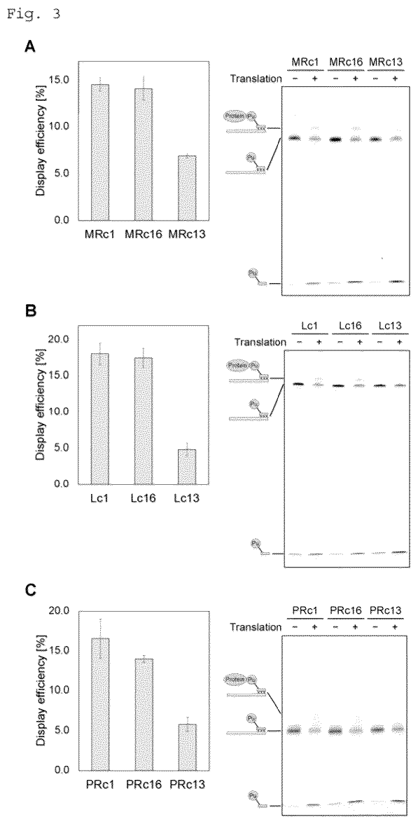

First, the enrichment factor was calculated. FIG. 4 shows a heat map in which the enrichment factor of each C-terminal sequence in the Monobody library is expressed by color. A figure was prepared by plotting the enrichment factor of each C-terminal sequence in Monobody and Anticalin (FIG. 2A). The enrichment factor of each C-terminal sequence was strongly correlated (R²=0.83) between the library where the upstream sequence was Monobody and the library where the upstream sequence was Anticalin. From this result, it was suggested that a C-terminal sequence showing high display efficiency can be obtained in a versatile manner.

As targets of display efficiency measurement for confirming the correlation between the enrichment factor and the display efficiency, 19 types of C-terminal sequences showing various enrichment factors were selected (Table 3), DNA in which these sequences were introduced into the C-terminus of Monobody was prepared and transcribed into mRNA (designated as McX). The transcribed mRNA was reacted in a TRAP reaction system, and the display efficiency was measured. As a result, it was found that the enrichment factor in a library having Monobody as an upstream sequence strongly correlates with the display efficiency (R²=0.83) (FIG. 2B). That is, it was found that the display efficiency indicated by the C-terminal sequence can be indirectly evaluated from the index of the enrichment factor calculated by this method.

TABLE 3

| | Sequence | | | | | | | | | |
|---|---|---|---|---|---|---|---|---|---|---|
| | Codon 1 | | Codon 2 | | Base 1 | Base 2 | Enrichment (Monobody) | | Enrichment ( [symbol] ) | |
| Name | DNA | AA | DNA | AA | DNA | DNA | Value | Ranking | Value | Ranking |
| c1 | GGC | G | CGA | R | T | — | 4.23 | 1 | [symbol] | 1 |
| c17 | GSC | G | CGA | R | — | — | 4.21 | 2 | [symbol] | 13 |
| c2 | GGC | G | CGA | R | C | — | [symbol] | 3 | [symbol] | 40 |
| c3 | CGT | R | CGA | R | T | — | [symbol] | 4 | 2.99 | 34 |
| c4 | [symbol] | G | AGC | S | C | — | 3.81 | 5 | 2.82 | 55 |
| c5 | [symbol] | G | AGA | R | C | — | 3.79 | [symbol] | 3.50 | 4 |
| c6 | [symbol] | G | AGA | R | T | — | 3.75 | 7 | 3.74 | 2 |
| c18 | CGT | R | CGA | R | — | — | [symbol] | 9 | 2.95 | 38 |
| c7 | [symbol] | G | ACA | T | C | — | [symbol] | [symbol] | [symbol] | 32 |
| c8 | GGT | G | AGC | S | C | — | 3.17 | [symbol] | [symbol] | 54 |
| c19 | GGC | G | [symbol] | P | C | — | [symbol] | [symbol] | [symbol] | [symbol] |
| c9 | GGT | G | GCT | A | C | — | 2.80 | 87 | 2.55 | 101 |
| c [symbol] | [symbol] | G | AGC | S | — | — | 2.42 | [symbol] | [symbol] | 150 |
| c10 | [symbol] | A | [symbol] | [symbol] | C | — | 2.38 | 225 | [symbol] | [symbol] |
| c13 | GAC | D | AGC | S | C | — | 2.02 | [symbol] | [symbol] | 1113 |
| c12 | CGT | R | CGC | R | [symbol] | — | 1.49 | 1179 | 1.25 | [symbol] |
| c13 | [symbol] | P | [symbol] | R | C | — | 1.00 | [symbol] | [symbol] | [symbol] |
| c14 | AGC | S | [symbol] | N | C | — | [symbol] | [symbol] | [symbol] | [symbol] |
| c15 | CGG | R | GAA | E | [symbol] | — | 0.30 | 7052 | [symbol] | [symbol] |

[symbol]

indicates data missing or illegible when filed

The sequence of Codon 1 having an enrichment factor of 3.2 or more in at least one of Monobody and Anticalin was GGC, CGT, or GGT, and the sequence of Codon 2 having an enrichment factor of 3.2 or more in at least one of Monobody and Anticalin was CGA, AGC, AGA, ACA, CGT, AGT, GCA, AGG, GGT, CGC, GCT, GGC, CGG, ACT, GAT, or GAC. The sequence of Codon 1-Codon 2 having an enrichment factor of 2.8 or more in both Monobody and Anticalin was GGCCGA, GGCAGA, GGCGCA, GGTAGA, GGCAGT, GGCAGC, GGCACA, GGCCGT, GGCGCT, GGCGAT, GGCACT, GGTCGA, GGCAGG, GGCACG, GGTGAC, GGTAGG, CGTCGA, GGTAGT, GGTCGT, GGCGGT, GGACGA, GGTGGT, GGTACA, GGTCGC, AATCGA, GGTAGC, or GCGCGA. The sequence of Codon 1-Codon 2 having an enrichment factor of 3.0 or more in both Monobody and Anticalin was GGCCGA, GGCAGA, GGTAGA, GGCACA, GGCCGT, GGCAGT, GGCAGC, GGCAGG, GGTCGA, GGCGCT, GGCACT, GGCGCA, or GGTAGG. The sequence of Codon 1-Codon 2 having an enrichment factor of 3.2 or more in both Monobody and Anticalin was GGCCGA, GGCAGA, GGTAGA, GGCCGT, GGCAGT, GGCAGC, GGTCGA, GGCGCT, or GGCACT. A polynucleotide in which a polypeptide encoding sequence or a polypeptide encoding sequence insertion site, the Codon 1, the Codon 2, and the stop codon are arranged in this order and the Codon 1 and the Codon 2 are adjacent to each other can exhibit high display efficiency in a polynucleotide display method.

### Test Example 2. Evaluation of Display Efficiency with High Versatility of Obtained C-Terminal Sequence

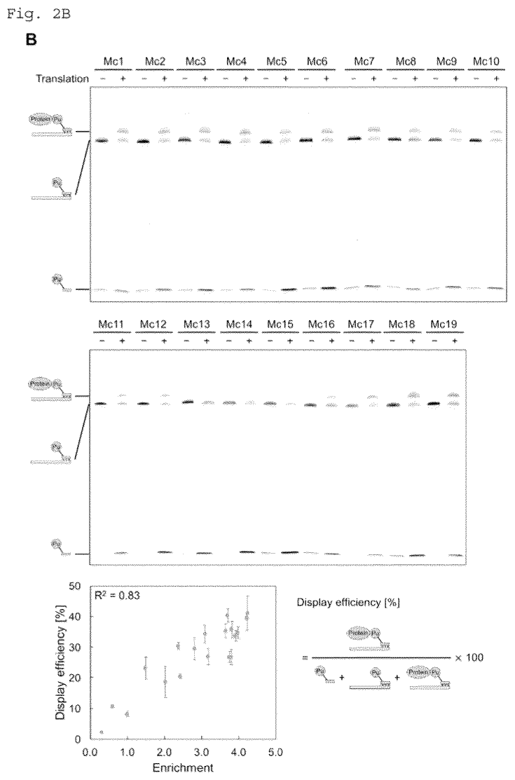

Previous experiments used Monobody WT, which has a constant overall sequence. However, when functional molecules are actually obtained by an evolutionary molecular engineering technique, Monobody library having great diversity by randomizing two loop structures as binding sites is used. Therefore, it cannot be said that an effective sequence is obtained unless high display efficiency is shown not only for Monobody WT but also for Monobody library including a random sequence. Thus, it was checked whether the obtained C-terminal sequence is also applicable to Monobody library. Monobody library used in this study was designed and prepared with reference to previous studies. Monobody library has a random sequence in two loop portions, and the random sequence is designed not to include a stop codon. DNA in which c1 at the first enrichment factor, c16 at the 194th position, and c13 at the 2448th position were introduced at the C-terminus of Monobody library was prepared and transcribed into mRNA (designated as MRCX). The transcribed mRNA was reacted in a TRAP reaction system at the optimized time, and the display efficiency was measured. As a result, MRc1 and MRc16 included in the top 3% of the enrichment factor had significantly higher display efficiency than MRc13 (MRc1: 14.6%, MRc16: 14.1%, MRc13: 6.91%) (FIG. 3A). Therefore, it can be said that when a C-terminal sequence having an enrichment factor within the top 3% is selected, high display efficiency is exhibited even for a library having a random sequence at the binding site.

However, the high display efficiency also for Monobody library may be caused by 78 to 84% homology with Monobody WT. Therefore, DNA in which three types of C-terminal sequences similar to those described above were introduced into the C-terminus of Anticalin, which is another artificial antibody skeleton having no homology between sequences at all, was prepared and transcribed into mRNA (designated as LcX). The transcribed mRNA was reacted in a TRAP reaction system, and the display efficiency was measured. As a result, Lc1 and Lc16 included in the top 3% of the enrichment factor had significantly higher display efficiency than Lc13 (Lc1: 18.0%, Lc16: 17.4%, Lc13: 4.85%) (FIG. 3B). Therefore, it can be said that when a C-terminal sequence having an enrichment factor within the top 3% is selected, high display efficiency is exhibited even for other artificial antibody skeletons having completely different sequences.

Next, it was checked whether the C-terminal sequence obtained in this screening can also be applied to a macrocyclic peptide library expected as a new drug discovery modality. The macrocyclic peptide library used in this study has a random sequence of 15 residues, and N-chloroacetyl-L-phenylalanine and cysteine at both ends thereof are adapted to cause a cyclization reaction. The random sequence is designed to be free of a stop codon. DNA in which three types of C-terminal sequences similar to those described above were introduced into the C-terminus of the macrocyclic peptide library was prepared and transcribed into mRNA (designated as PRCX). The transcribed mRNA was reacted in a TRAP reaction system, and the display efficiency was measured. As a result, PRc1 and PRc16 had significantly higher display efficiency than PRc13 (PRc1: 16.5%, PRc16: 14.0%, PRc13: 5.79%) (FIG. 3C). Therefore, it can be said that when a C-terminal sequence having an enrichment factor within the top 3% is selected, high display efficiency is exhibited even for the macrocyclic peptide library.

### Test Example 3. Screening for N-Terminal Random Sequence

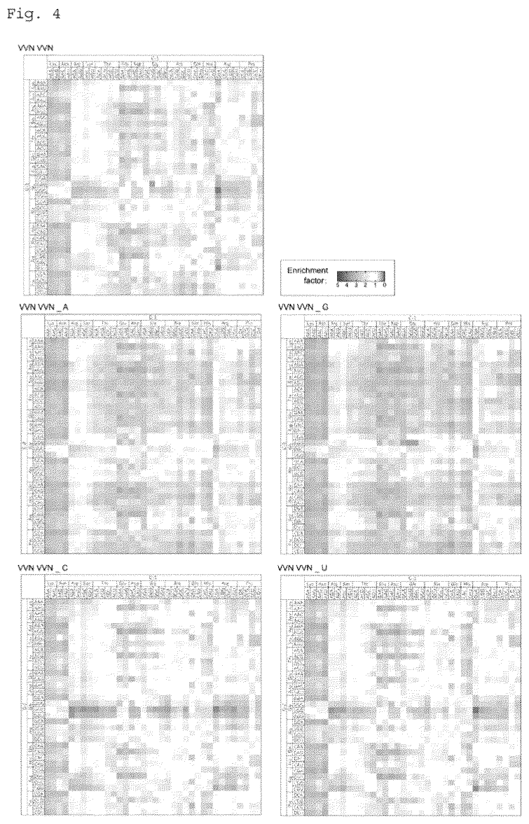

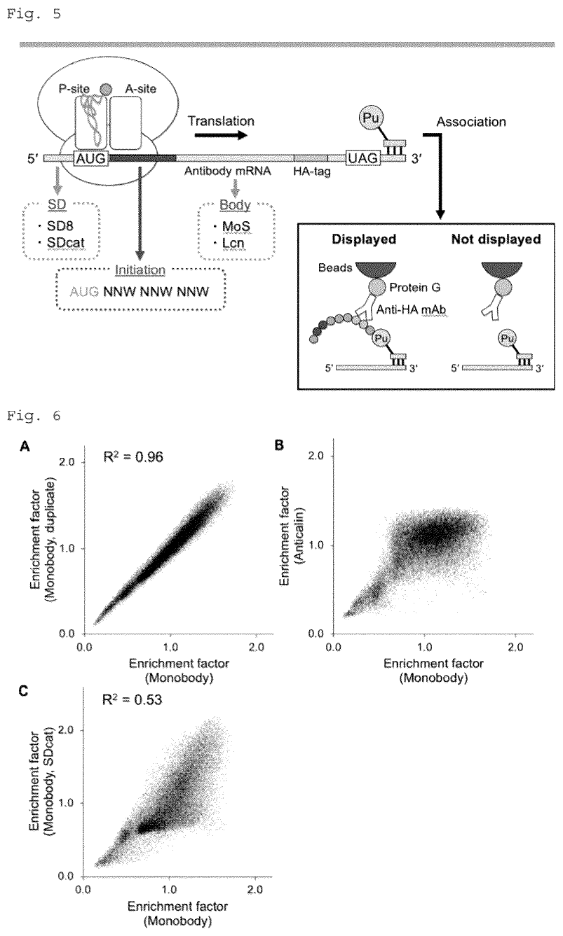

For the N-terminal random sequence adjacent to AUG, the enrichment factor was also measured in the same manner as screening for the C-terminal random sequence (FIGS. 5 and 6). As a result, as in the screening for the C-terminal random sequence, it was found that the enrichment factor varies depending on the sequence, and there is a sequence with a high enrichment factor. When two types of Monobodies having different upstream sequences including the SD sequence were compared, it was found that the enrichment factor depends on the combination of the SD sequence and the N-terminal random sequence. When Monobody and Anticain having an upstream sequence including the same SD sequence were compared, it was found that the enrichment factor depends on the combination of the protein and the N-terminal random sequence, The enrichment factor here reflects the efficiency of translation initiation, which is important in protein expression. Therefore, by obtaining the enrichment efficiency for any protein by this method, it is possible to obtain a sequence group capable of controlling the expression of any protein. This information allows precise control of protein expression, and is considered to be useful for highly efficient production of proteins.

### Test Example 4. Data Analysis and Evaluation 2 of Screening Results for C-Terminal Random Sequence

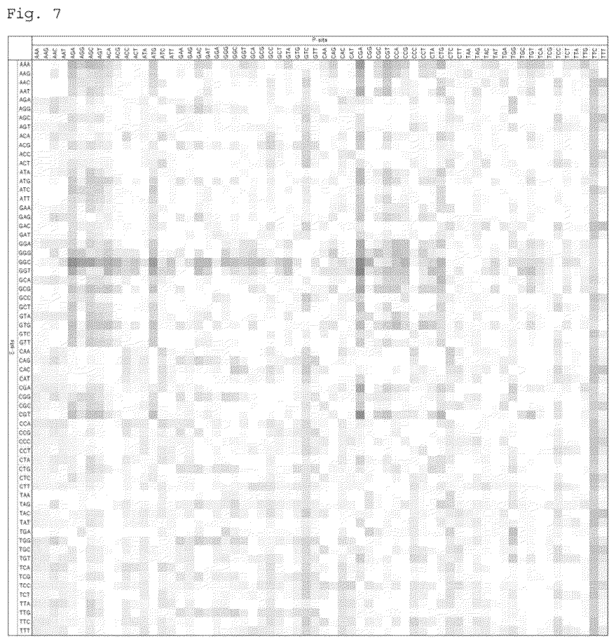

A mixed DNA library was prepared in which a C-terminal random sequence of type I (NNNNNNTAG) was added to an upstream sequence (Monobody). Screening was performed using the library in the same manner as in Test Example 1, and the enrichment factor was calculated. FIG. 7 shows a heat map in which the enrichment factor of each C-terminal sequence in the Monobody library is expressed by color. The correspondence relationship between the color and the enrichment factor is the same as that in FIG. 4.

The sequence of Codon 1 having a relatively high enrichment factor was AAA, GGC, CGT, or GGT, and the sequence of Codon 2 having a relatively high enrichment factor was ACA, AGA, AGC, AGG, AGT, ATG, CCA, CCG, CCT, CGA, CGC, CGT, CTA, or CTG.

### Sequence Listing

1. A method for screening for a sequence that regulates display efficiency in a polynucleotide presentation method, the screening method comprising steps of:
(a) binding a polynucleotide-polypeptide complex, which is obtained through translation by a cell-free translation system from a polynucleotide including a polypeptide encoding sequence and random sequences, to a solid phase via a modification substance on the polypeptide in the polynucleotide-polypeptide complex; and
(b) selecting a random sequence by using, as an index, an enrichment factor obtained by measuring and calculating a ratio (enrichment factor) of appearance frequency of each random sequence in the polynucleotide-polypeptide complex bound to the solid phase with respect to appearance frequency of each random sequence in the polynucleotide.

2. The screening method according to claim 1, wherein in step (b), a random sequence having a high enrichment factor is selected as a sequence that improves display efficiency.

3. The screening method according to claim 1, wherein in step (b), a random sequence having the top 10% of the enrichment factor is selected as a sequence that improves display efficiency.

4. The screening method according to claim 1, wherein the modification substance is at least one selected from the group consisting of biotin, a peptide tag, and a substance containing a readily reactive group.

5. The screening method according to claim 1, wherein the random sequence is arranged downstream side of the polypeptide encoding sequence.

6. The screening method according to claim 5, wherein a linker sequence is arranged between the random sequence and the polypeptide encoding sequence.

7. The screening method according to claim 5, wherein a stop codon is arranged adjacent to the random sequence or in the random sequence.

8. A reagent for a polynucleotide presentation method, the reagent comprising a polynucleotide, wherein the polynucleotide is a polynucleotide including a polypeptide encoding sequence or a polypeptide encoding sequence insertion site, a sequence 2 having 3 bases in length, a sequence 2 having 3 bases in length, and a stop codon, the polypeptide encoding sequence or polypeptide encoding sequence insertion site, the sequence 1, the sequence 2, and the stop codon being arranged in this order from upstream, the sequence 1 being adjacent to the sequence 2, the sequence 2 being adjacent to the stop codon, and the sequence 1 being GGC, CGT, GGT, or AAA, and/or the sequence 2 being CGA, AGC, AGA, ACA, CGT, AGT, GCA, AGG, GGT, CGC, GCT, GGC, CGG, ACT, GAT, GAC, ATG, CCA, CCG, CCT, CTA, or CTG, and
the polynucleotide presentation method is a polynucleotide presentation method using a cell-free translation system which is a prokaryotic cell translation system.

9. The reagent according to claim 8, wherein the sequence 1 is GGC, CGT, GGT, or AAA, and the sequence 2 is CGA, AGC, AGA, ACA, CGT, AGT, GCA, AGG, GGT, CGC, GCT, GGC, CGG, ACT, GAT, GAC, ATG, CCA, CCG, CCT, CTA, or CTG.

10. The reagent according to claim 8, wherein a contiguous sequence of the sequence 1 and the sequence 2 is GGCCGA, GGCAGA, GGCGCA, GGTAGA, GGCAGT, GGCAGC, GGCACA, GGCCGT, GGCGCT, GGCGAT, GGCACT, GGTCGA, GGCAGG, GGCACG, GGTGAC, GGTAGG, CGTCGA, GGTAGT, GGTCGT, GGCGGT, GGACGA, GGTGGT, GGTACA, GGTCGC, AATCGA, GGTAGC, or GCGCGA.

11. (canceled)

12. (canceled)

13. (canceled)

14. The screening method according to claim 2, wherein in step (b), a random sequence having the top 10% of the enrichment factor is selected as a sequence that improves display efficiency.

15. The screening method according to claim 2, wherein the modification substance is at least one selected from the group consisting of biotin, a peptide tag, and a substance containing a readily reactive group.

16. The screening method according to claim 3, wherein the modification substance is at least one selected from the group consisting of biotin, a peptide tag, and a substance containing a readily reactive group.

17. The screening method according to claim 2, wherein the random sequence is arranged downstream side of the polypeptide encoding sequence.

18. The screening method according to claim 3, wherein the random sequence is arranged downstream side of the polypeptide encoding sequence.

19. The screening method according to claim 4, wherein the random sequence is arranged downstream side of the polypeptide encoding sequence.

20. The screening method according to claim 6, wherein a stop codon is arranged adjacent to the random sequence or in the random sequence.

21. The reagent according to claim 9, wherein a contiguous sequence of the sequence 1 and the sequence 2 is GGCCGA, GGCAGA, GGCGCA, GGTAGA, GGCAGT, GGCAGC, GGCACA, GGCCGT, GGCGCT, GGCGAT, GGCACT, GGTCGA, GGCAGG, GGCACG, GGTGAC, GGTAGG, CGTCGA, GGTAGT, GGTCGT, GGCGGT, GGACGA, GGTGGT, GGTACA, GGTCGC, AATCGA, GGTAGC, or GCGCGA.
